(19) United States
(12) Patent Application Publication
(10) Pub. No.: US 2026/0355461 A1
(43) Pub. Date: Nov. 12, 2026

(54) SYSTEMS AND METHOD AND DEVICES FOR TARGETED IV CATHETER PLACEMENT USING A ONE-HANDED DEVICE

(71) Applicant: Zachary D. Walton, Gloucester, MA (US)

(72) Inventor: Zachary D. Walton, Gloucester, MA (US)

(21) Appl. No.: 19/665,292

(22) Filed: May 1, 2026

Related U.S. Application Data
(60) Provisional application No. 63/800,541, filed on May 6, 2025.
(60) Provisional application No. 63/800,532, filed on May 6, 2025.

(51) Int. Cl. A61M 25/01; A61M 25/06

(52) U.S. Cl. CPC A61M 25/0113; A61M 25/0606; A61M 2205/586

(57) ABSTRACT

Aspects of the present disclosure describe medical device placement, including devices, systems and methods for placing other medical devices, such as needles and catheters using a one-handed device.

### CROSS-REFERENCE TO RELATED APPLICATIONS

The present application claims the benefit of U.S. Provisional Patent Application Ser. No. 63/800,541, filed May 6, 2025 and U.S. Provisional Patent Application Ser. No. 63/800,532, filed May 6, 2025, the entire contents of which are specifically incorporated herein by reference.

### FIELD

The present technology is generally related to medical device placement, for example, needles, catheters, etc., using an assistive medical device for such placement.

### BACKGROUND

Placement of a catheter in a vessel relies upon accurate placement of a needle in the vessel prior to pushing the catheter overlying the needle into the vessel. Once the catheter is successfully in place, the needle can be withdrawn and the catheter can be used in place.

Accurately positioning the needle can be done via a variety of techniques, with or without various assistive devices, such as ultrasound probes, etc.

The present disclosure recognizes that use of needle placement in conjunction with real-time ultrasound guidance can involve complex or bulky components that can be difficult to coordinate for accurate placement and that typically require multiple operators to effectively provide placement by attempting to maintain proper visualization of the image as the needle is positioned.

Further, once the needle is in place in the vessel, care must be taken to maintain the position of the needle tip within the lumen of the vessel as the catheter is advanced over the needle so that the catheter cannulates the vein. It is important that nothing other than the catheter moves while the catheter is being advanced, because if the needle tip moves by a fraction of a millimeter, the catheter may be placed outside the vein.

One approach for achieving precision control while advancing the catheter is for the clinician to set the ultrasound aside and to use the non-dominant hand to advance the catheter into the vessel while holding the needle still with the dominant hand. The drawback to this technique is that the catheter is not visualized in the vein as the catheter is advanced, reducing the probability of success. All existing methods of advancing the catheter with only the dominant hand rely on repositioning the grip or on placing forces on the catheter-needle assembly that risk dislodging the catheters.

Accordingly, the present disclosure recognizes that there is a need in the art for improvements in ease, reliability, and repeatability of medical device placement. This disclosure introduces devices and methods for using ultrasound for real-time guidance of medical device placement for needle placement that overcomes the above-described and other challenges and problems in the art.

### SUMMARY

The techniques of this disclosure generally relate to medical device placement, including devices, systems and methods for placing catheters in vessels by providing a device allowing a clinician's use of a dominant hand to advance the catheter with minimal repositioning. In exemplary aspects, the one-handed device includes a handle and a needle-catheter assembly holder that is advanceable in a single direction along a plane via an activation member, such as an integral ring or prong, to slide the catheter over the needle and into place.

In further exemplary aspects, a loading template arranges the needle-catheter assembly in the device such that the needle is held in place with the catheter thereover, but that advancing the activation member only advances the catheter portion of the needled/catheter assembly. In exemplary embodiments, this is done using a finger, e.g., an index finger, to advance the catheter. No extraneous forces are applied to the needle-catheter assembly. In further exemplary embodiments, a quick release mechanism releases the needle from the handle for disposal in a sharps container.

The one-handed nature of the device ensures that the clinician can use a dominant hand even while, e.g., maintaining visualization using an ultrasound in the non-dominant hand. Additionally, the clinician maintains a greater distance from the tip of the needle during the placement process as compared to existing techniques. This increases the safety margin for the patient and for the clinician.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of a first exemplary embodiment of a one-handed catheter advancing device, in accordance with exemplary aspects of the present disclosure;

FIG. 2 is a rear elevation view of the distal end of the exemplary device of FIG. 1, in accordance with exemplary aspects of the present disclosure;

FIG. 3 is a front elevation view of the exemplary device of FIG. 1, in accordance with exemplary aspects of the present disclosure;

FIG. 4 is a top elevation view of the exemplary device of FIG. 1, in accordance with exemplary aspects of the present disclosure; and

FIG. 5 is another perspective view of the exemplary device of FIG. 1, in accordance with exemplary aspects of the present disclosure.

### DETAILED DESCRIPTION

As we have noted, the techniques of this disclosure generally relate to medical device placement, including devices, systems and methods for placing catheters with precision and improved safety.

In exemplary aspects, the techniques and exemplary devices of this disclosure provide medical device placement, including devices, systems and methods for placing catheters in vessels by providing a device allowing a clinician's use of a dominant hand to advance the catheter with minimal repositioning. In exemplary aspects, the one-handed device includes a handle and a needle-catheter assembly holder that is advanceable in a single direction along a plane via an activation member, such as an integral ring or prong, to slide the catheter over the needle and into place.

In further exemplary aspects, a loading template arranges the needle-catheter assembly in the device such that the needle is held in place with the catheter thereover, but that advancing the activation member only advances the catheter portion of the needled/catheter assembly. In exemplary embodiments, this is done using a finger, e.g., an index finger, to advance the catheter. No extraneous forces are applied to the needle-catheter assembly. In further exemplary embodiments, a quick release mechanism releases the needle from the handle for disposal in a sharps container.

The one-handed nature of the device ensures that the clinician can use a dominant hand even while, e.g., maintaining visualization using an ultrasound in the non-dominant hand. Additionally, the clinician maintains a greater distance from the tip of the needle during the placement process as compared to existing techniques. This increases the safety margin for the patient and for the clinician.

In further exemplary aspects, a device is provided that facilitates single-operator procedures with real-time ultrasound guidance. The exemplary device allows the operator to place the needle with (e.g.) millimeter precision with one needle pass and complete the procedure without assistance, advancing only the catheter into the vessel using one hand.

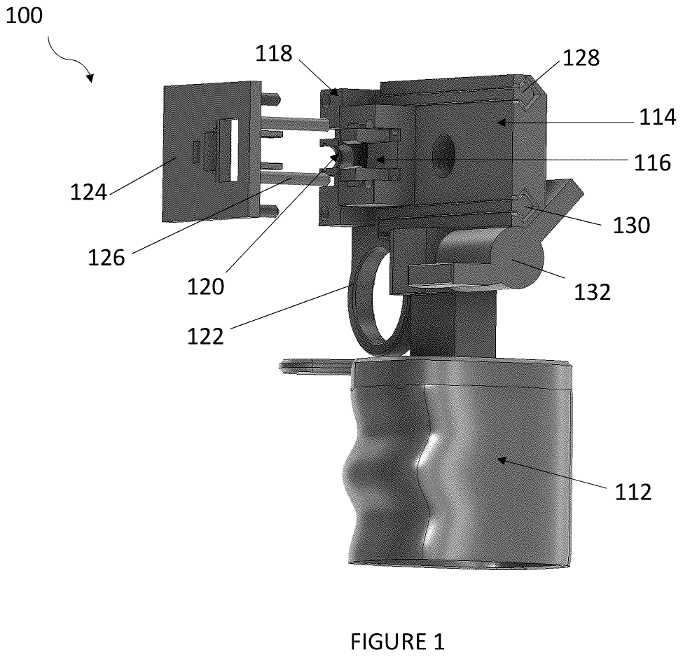

Referring now to FIG. 1, an exemplary one-handed catheter placement device is illustrated in perspective generally at 100. The exemplary device includes a handle 112, a stationary needle tray 114 configured to securely hold the needle portion of a needle-catheter assembly, e.g., in needle mount portion 116, a catheter advancing tray 118 including a catheter engaging portion 120 configured to push the catheter hub forward as activating mechanism (finger ring) 122 is pushed forward, and an alignment mechanism 124, configured place the needle portion in the needle holder and the catheter hub in the catheter engaging portion when pressed into position using plural guide posts 126. As can be seen, the catheter advancing tray 118 slides forward in two grooves 128, 130 configured to provide precise forward movement in a single plane. A quick release device is also provided at 132 to separate the needle holder from the handle for sharps disposal.

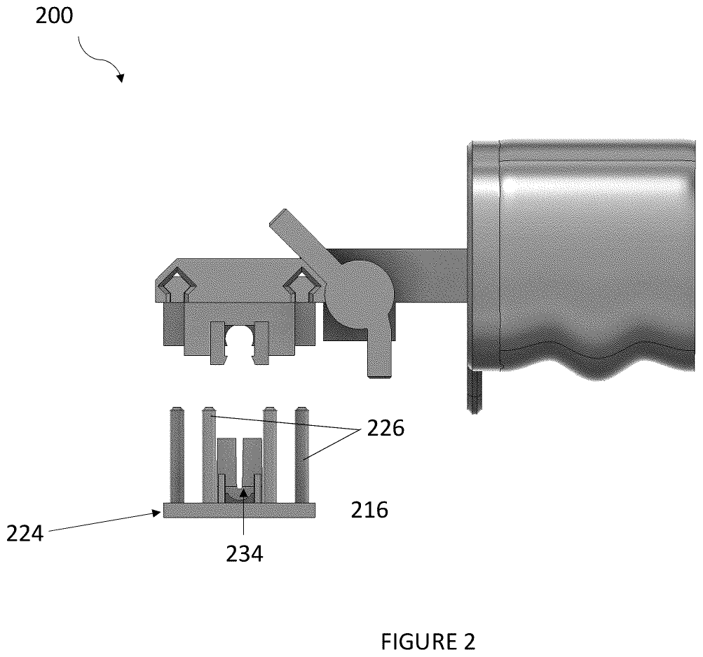

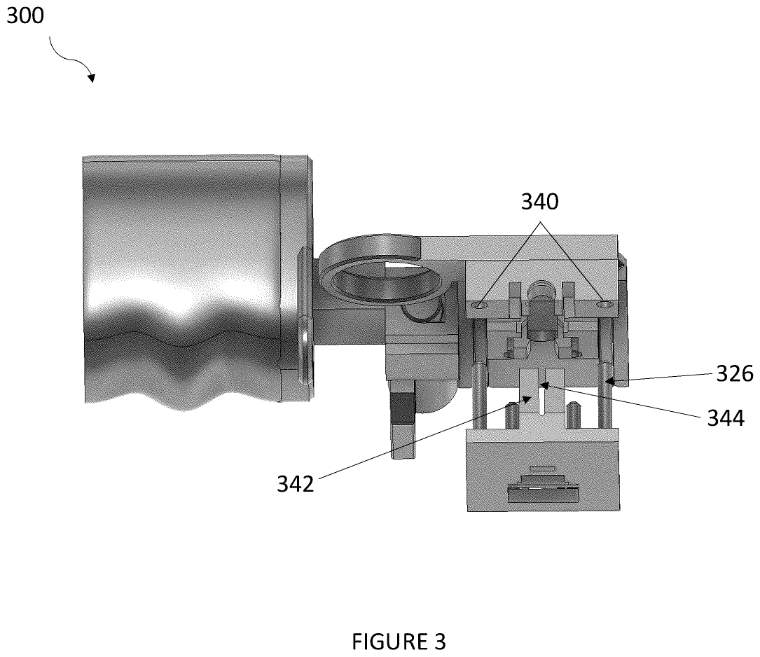

Referring now to FIG. 2, a rear elevational view is illustrated generally at 200 and better shows the alignment mechanism/tray with four posts 226 that are received in slots in the device for alignment. Alignment mechanism 224 needle guide 234 is also illustrated. FIG. 3 additionally provides a front elevation view at 300 that additionally shows alignment holes 340 complementary to posts 326. A front alignment member 342 includes a slot 344 to allow the needle and catheter to protrude during installation. In exemplary embodiments, the alignment mechanism is removed after installation of the needle-catheter assembly.

While FIG. 3 shows an alignment mechanism used to install the needle and catheter, it should be recognized that exemplary embodiments described herein also include a fully assembled or at least partially integrated device, e.g., a medical device that is provided by the manufacturer in sterile packaging ready for use (with or without inclusion of the illustrated alignment mechanism), such that the needle is attached to the handle and the catheter, which overlies the needle is attached to the slidable actuation member (which may or may not include the illustrated ring member), such that the catheter can slide over the needle and into position for use with a patient.

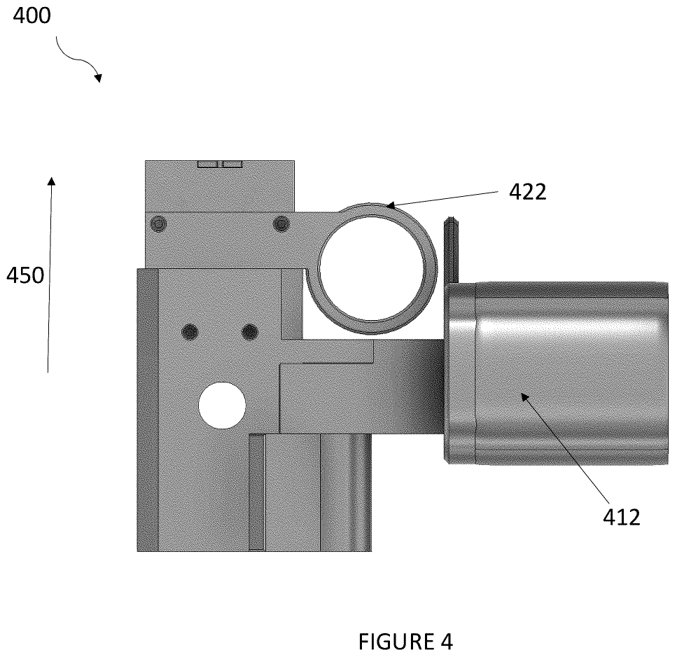

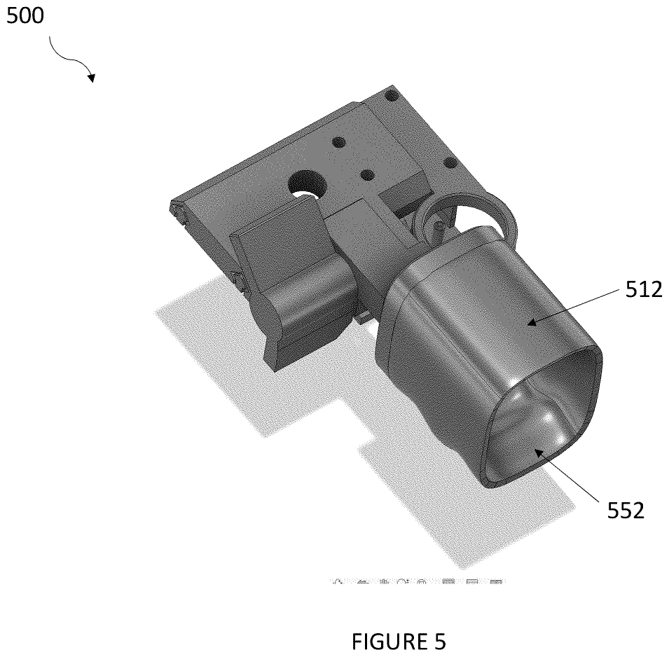

FIG. 4 shows a top elevation view generally at 400, as a clinician might grip it, e.g., with a right hand, with the handle 412 nesting plural fingers and the ring/activation mechanism 422 being slidable in the direction of arrow 450. FIG. 5 shows another perspective view, further illustrating e.g., ergonomic contours 552 on the lower portion of handle 512.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A one-handed medical device, comprising:
a handle;
a needle holding component secured to the handle; and
a catheter advancing component configured to actuate along a plane via an activation member to push a catheter over the secured needle using the same hand.

2. A device in accordance with claim 1, wherein the catheter advancing component is configured to advance in a single direction via the actuation member.

3. A device in accordance with claim 2, wherein the catheter advancing component slides within two tracks for catheter advancing motion along a plane.

3. A device in accordance with claim 1, further comprising an alignment mechanism configured to install a needle-catheter assembly such that the needle is secured in a non-movable fashion relative to the handle and such that the catheter advancing component is configured to act on the hub of the catheter to advance it over the needle.

4. A device in accordance with claim 3, wherein the alignment mechanism is configured to maintain the needle and catheter in proper installation alignment and to press the needle and catheter into place such that the needle engages and is held by the handle and the catheter is engaged by the catheter advancing component.

5. A device in accordance with claim 1, wherein the activation member is a ring or prong integral to the catheter advancing component and configured to provide one handed advancement of the catheter over the needle secured to the handle.

6. A device in accordance with claim 1, further comprising a loading template component configured to arrange the catheter and needle in place for securing the needle to the handle and securing the catheter to the catheter advancing component.

7. A device in accordance with claim 6, wherein the needle includes a mount portion configured to be securely retained in a stationary needle tray.

8. A device in accordance with claim 3, wherein the alignment mechanism includes plural guide posts configured to precisely install the needle and catheter assembly.

9. A device in accordance with claim 8, wherein the alignment mechanism includes four posts that are received in four slots in one or both of the catheter advancing component and handle.

10. A device in accordance with claim 9, wherein the alignment mechanism is configured with removable posts such that the alignment mechanism is removed prior to installation of the needle and catheter assembly, with the needle retaining engagement with the handle and with the catheter retaining engagement with the catheter advancing component.

11. A device in accordance with claim 1, further comprising a quick release mechanism attached to the handle and configured to release the needle from the handle for disposal in a sharps container.

12. A device in accordance with claim 1, wherein the handle includes ergonomic contours on a lower portion thereof.
